SEQUENCE LISTING (5 sequences)

SEQ ID NO: 1 - DNA, 10 bp, Artificial Sequence
  Description of Artificial Sequence Synthetic oligonucleotide
taccggttag

SEQ ID NO: 2 - DNA, 10 bp, Artificial Sequence
  Description of Artificial Sequence Synthetic oligonucleotide
taatgagttt

SEQ ID NO: 3 - DNA, 19 bp, Artificial Sequence
  Description of Artificial Sequence Synthetic oligonucleotide
taatacgactcactatagg

SEQ ID NO: 4 - DNA, 708 bp, Artificial Sequence
  Description of Artificial Sequence Synthetic polynucleotide
gtgagcaagggcgaggagaccacaatgggcgtaatcaagcccgacatgaagatcaagctgaagatggagggcaacgtgaatggccacgccttcgtgatcgagggcgagggcgagggcaagccctacgacggcaccaacaccatcaacctggaggtgaaggagggagcccccctgcccttctcctacgacattctgaccaccgcgttcagttacggcaacagggccttcaccaagtaccccgacgacatccccaactacttcaagcagtccttccccgagggctactcttgggagcgcaccatgaccttcgaggacaagggcatcgtgaaggtgaagtccgacatctccatggaggaggactccttcatctacgagatacacctcaagggcgagaacttcccccccaacggccccgtgatgcagaaggagaccaccggctgggacgcctccaccgagaggatgtacgtgcgcgacggcgtgctgaagggcgacgtcaagatgaagctgctgctggagggcggcggccaccaccgcgttgacttcaagaccatctacagggccaagaaggcggtgaagctgcccgactatcactttgtggaccaccgcatcgagatcctgaaccacgacaaggactacaacaaggtgaccgtttacgagatcgccgtggcccgcaactccaccgacggcatggacgagctgtacaagtaa

SEQ ID NO: 5 - DNA, 1653 bp, Artificial Sequence
  Description of Artificial Sequence Synthetic polynucleotide
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

(19) United States
(12) Patent Application Publication
(10) Pub. No.: US 2023/0111107 A1
(43) Pub. Date: Apr. 13, 2023

(54) METHOD FOR PREPARING MRNA-GALNAC TARGETING MOLECULE, IN VIVO DELIVERY SYSTEM THEREFOR, AND USE THEREOF

(71) Applicant: ShenZhen Rhegen Biotechnology Co., Ltd., ShenZhen (CN)

(72) Inventors: Yong Hu, Shenzhen (CN); Miaomiao Zhang, Shenzhen (CN)

(21) Appl. No.: 17/843,162

(22) Filed: Jun. 17, 2022

Related U.S. Application Data
(63) Continuation of application No. PCT/CN2020/135203, filed on Dec. 10, 2020.

(30) Foreign Application Priority Data
Jan. 10, 2020 (CN) 202010027183.1

(51) Int. Cl. A61K 48/00; C12N 15/88; C12N 15/11; A61K 47/54; C12P 19/34

(52) U.S. Cl. CPC A61K 48/0041; C12N 15/88; C12N 15/11; A61K 47/549; C12P 19/34; C12N 2310/351; C12N 2330/50; C12N 2320/32

(57) ABSTRACT

Provided are a method for preparing an mRNA-GalNAc targeting molecule, an in vivo delivery system therefor and use thereof. The mRNA-GalNAc targeting molecule comprises an mRNA molecule that is linked to PolyA modified with an N-acetylgalactosamine at 3′-end, wherein a sequence of the mRNA molecule comprises a 5′ cap and a target gene sequence. By directly linking the mRNA molecule expressing the target gene to the polyA sequence coupled with GalNAc, an mRNA molecule with GalNAc at 3′-end is synthesized to realize the aim of targeted drug delivery in liver. The method is simpler and more reliable, and solves existing problems in coupling between mRNA and N-acetylgalactosamine.

The present application claims priority of Chinese Patent Application No. CN202010027183.1, entitled “METHOD FOR PREPARING MRNA-GALNAC TARGETING MOLECULE, IN VIVO DELIVERY SYSTEM THEREFOR, AND USE THEREOF” and filed with China National Intellectual Property Administration on Jan. 10, 2020, the entire content of which is incorporated by reference herein.

### TECHNICAL FIELD

The present invention belongs to the technical field of molecular biology, and in particular relates to a method for preparing an mRNA-GalNAc targeting molecule, an in vivo delivery system therefor and use thereof.

### BACKGROUND OF THE INVENTION

At present, delivering mRNAs into cells can be achieved by different methods, such as electroporation, sonoporation, microinjection, or compound transfection. However, these methods cannot meet clinical needs in term of cytotoxicity and biological safety, and have certain difficulties in clinical transformations.

An asialoglycoprotein receptor (ASGPR) is an abundant hetero-oligomer endocytic receptor, mainly exists on a surface of cell membrane of liver parenchymal cells facing the sinusoid, and has specificity to sugar. Since exposed secondary ends are galactose residues after terminal sialic acids of various glycoproteins are removed via enzyme hydrolysis or acidolysis, the sugar-binding specificity of ASGPR actually acts on galactosyl groups and ASGPR is also called as a galactose specific receptor. Glycoproteins with non-reductive galactose (Gal) or N-acetylgalactosamine (GalNAc) residues at the ends can be recognized by ASGPR, and the affinity of ASGPR binding to GalNAc is dozens of times (about 50 times) higher than that to Gal. It has been observed in studies that the tri-tentacle clustered GalNAc conjugates can increase the affinity of GalNAc with hepatic parenchymal cells by about 50 times.

After years of continuous research and development, the GalNAc-conjugated small interfering RNA (siRNA) has achieved high-efficiency targeted drug delivery in liver. Although the receptor has been discovered for many years, messenger RNA (mRNA) delivery systems based on the receptor and its ligands have failed to achieve a breakthrough because of problems such as failure to achieve high-efficiency coupling between GalNAc and mRNA by existing technical means, charge property of the mRNA-GalNAc conjugate during in vivo delivery, and efficiency of escape from endosome after endocytosed by a cell.

### SUMMARY OF THE INVENTION

In view of the above technical problems, the present invention discloses a method for preparing an mRNA-GalNAc targeting molecule, in vivo delivery system therefor and use thereof, realizing direct and high-efficiency coupling between mRNA and N-acetylgalactosamine, modifying charge property of the mRNA-GalNAc conjugate during in vivo delivery by a positively charged protein, and increasing its efficiency of escape from endosome.

For this, the technical solutions adopted by the present invention are as follows.

The present invention provides an mRNA-GalNAc targeting molecule, comprising an mRNA molecule that is linked to PolyA modified with an N-acetylgalactosamine at 3′-end, wherein a sequence of the mRNA molecule comprises a 5′ cap and a target gene sequence.

Preferably, the sequence of the mRNA molecule further comprises 5′UTR and 3′UTR, wherein the 5′UTR comprises a Kozak sequence.

In the present invention, the sequence of the mRNA is formed by sequentially connecting the 5′cap, the 5′UTR sequence comprising the Kozak sequence, the target gene sequence, the 3′UTR sequence and the PolyA sequence.

Preferably, the mRNA molecule is consisted of uridine, cytosine, adenosine, guanosine and a chemically modified nucleoside, wherein the chemically modified nucleoside is selected from one or more of 2-fluoro-2-deoxyadenosine, 2-fluoro-2-deoxyuridine, 2-fluoro-2-deoxycytidine, 2-fluoro-2-deoxyguanosine, 2-fluoro-2-deoxy-5-methylcytidine, 2-fluoro-2-deoxy-pseudouridine, 2-fluoro-2-deoxy-N1-methyl-pseudouridine, 2-fluoro-2-deoxy-N7-methyl-guanosine, 2-fluoro-2-deoxy-5-methoxyuridine, 2-fluoro-2-deoxy-N4-acetylcytidine, 2-fluoro-2-deoxy-N6-methyladenosine, 5-methylcytidine, pseudouridine, N1-methyl-pseudouridine, N7-methyl-guanosine, 5-methoxyuridine, N4-acetylcytidine and N6-methyladenosine.

Preferably, the 5′cap structure is selected from one or more of Cap0, Cap1, Cap2, ARCA, inosine, N1-methyl-guanosine, 2′fluoro-guanosine, 7-deaza-guanosine, 8-oxo-guanosine, 2-amino-guanosine, LNA-guanosine, 2-azido-guanosine, 7-methyl-guanosine-5′-triphosphate-5′-adenosine, guanosine-5′-triphosphate-5′-adenosine, 7-methyl-guanosine-5′-triphosphate-5′-guanosine, guanosine-5′-triphosphate-5′-guanosine, and 7-methyl-guanosine-5′-triphosphate-5′-2-methoxyadenine-guanosine.

Preferably, the mRNA molecule is linked to the PolyA modified with an N-acetylgalactosamine at 3′-end through a splint DNA.

Preferably, the mRNA molecule is obtained by in vitro transcribing a plasmid comprising a DNA fragment, wherein the DNA fragment comprises a promoter, a target gene, and a first splint DNA sequence that are sequentially connected; and the PolyA fragment modified with an N-acetylgalactosamine at 3′-end has a 5′-end which is an RNA sequence that can be complementarily paired with a second splint DNA sequence, wherein the first splint DNA sequence and the second splint DNA sequence form a splint DNA.

Preferably, the first splint DNA sequence is as shown in SEQ ID No. 1, the second splint DNA sequence is as shown in SEQ ID No. 2, and the promoter is a T3 or T7 or SP6 promoter.

The present invention further provides a method for preparing an mRNA-GalNAc targeting molecule as described above, comprising the following steps:

Step S1, designing and synthesizing a plasmid vector having a promoter sequence and a target gene sequence;

Step S2, in vitro transcribing the plasmid vector of Step S1 as a template to obtain an mRNA molecule, wherein a sequence of the mRNA molecule comprises a 5′cap and the target gene sequence; and

Step S3, binding the mRNA molecule to a PolyA sequence modified with an N-acetylgalactosamine at 3′-end under the action of a ligase, to obtain the mRNA-GalNAc targeting molecule.

Preferably, in Step S1, the DNA sequence in the plasmid vector comprises the promoter sequence, the target gene sequence, and a sequence complementary to a first splint DNA sequence, that are sequentially connected.

The sequence of the mRNA molecule obtained in Step S2 comprises the 5′cap, the target gene sequence, and an RNA sequence corresponding to the sequence complementary to the first splint DNA sequence, that are sequentially connected.

In Step S3, the PolyA fragment modified with an N-acetylgalactosamine at 3′-end has a 5′-end which is an RNA sequence that can be complementarily paired with a second splint DNA sequence, wherein the first splint DNA sequence and the second splint DNA sequence form a splint DNA. In annealing reaction, the mRNA molecule with the sequence complementary to the first splint DNA sequence and the PolyA molecule with the sequence complementarily paired with the second splint DNA sequence complementarily bind to the splint DNA, respectively. A 3′-end hydroxyl group of the mRNA molecule is linked to a 5′-end phosphate group of the PolyA modified with an N-acetylgalactosamine under the action of a T4 DNA ligase. After treatment by a DNase, the mRNA targeting molecule modified with an N-acetylgalactosamine is obtained.

Further, in Step S3, the PolyA fragment modified with an N-acetylgalactosamine at 3′-end has a 5′-end which is an RNA sequence that can be complementarily paired with a second splint DNA sequence, wherein the first splint DNA sequence and the second splint DNA sequence form the splint DNA; and

in the absence of the splint DNA, directly coupling the mRNA molecule to the PolyA modified with an N-acetylgalactosamine at 3′-end has problems of a high mismatch rate and low linking efficiency. The technical solution of the present invention adopts a splint DNA, which can greatly improve efficiency and accuracy of linking. In annealing reaction, the mRNA molecule with the sequence complementary to the first splint DNA sequence and the PolyA molecule with the RNA sequence complementarily paired with the second splint DNA sequence complementarily bind to the splint DNA, respectively. A 3′-end hydroxyl group of the mRNA molecule is linked to a 5′-end phosphate group of the PolyA modified with an N-acetylgalactosamine under the action of a T4 DNA ligase. After treatment by a DNase, the mRNA targeting molecule modified with an N-acetylgalactosamine is obtained.

Preferably, the promoter is a T3 or T7 or SP6 promoter.

Preferably, the first splint DNA sequence includes, but is not limited to, the sequence as shown in SEQ ID No. 1, specifically 5′-taccggttag-3′, and the second splint DNA sequence includes, but is not limited to, the sequence as shown in SEQ ID No. 2, specifically 5′-taatgagttt-3′.

The present invention further provides a pharmaceutical composition comprising the mRNA-GalNAc targeting molecule as described above and a pharmaceutically acceptable excipient.

The present invention further provides a use of the mRNA-GalNAc targeting molecule or the pharmaceutical composition as described above in preparation of a medicament for expressing a target polypeptide in a mammalian subject.

The present invention further provides an in vivo delivery system comprising the mRNA-GalNAc targeting molecule as described above and a positively charged protein molecule.

It has been found in researches that the directly coupled mRNA-GalNAc without charge neutralization cannot achieve tissue-targeting delivery in vivo, but can achieve only transfection to liver cells in an in vitro cell experiment. The delivery system of the present invention achieves in vivo delivery that does not rely on traditional liposomes and lipid nanoparticles, by using positively charged proteins, thereby improving the stability of mRNA-GalNAc in vivo, increasing the efficiency of escape from endosome after endocytosis, and achieving tissue and organ targeting delivery.

Preferably, the positively charged protein is at least one of protamine and human serum albumin. The technical solution of the present invention achieve tissue and organ targeting delivery in vivo by improving the stability of the mRNA-GalNAc in vivo by screening effective positively charged protein molecules and neutralizing the negative charge of the mRNA molecule itself. At present, applying positively charged proteins such as protamine and human serum albumin to nucleic acid delivery is mainly relied on coupling to vectors such as liposomes and lipid nanoparticles to realize their functions. The technical solutions of the present invention achieve tissue and organ targeting delivery in vivo that does not rely on traditional vectors such as liposomes and lipid nanoparticles by combining mRNA-GalNAc, protamine, and human serum albumin.

Preferably, the in vivo delivery system is consisted of protamine, human serum albumin and an mRNA targeting molecule modified with an N-acetylgalactosamine, and the molar ratio of protamine to human serum albumin is 1:(2.75-5.5) or 1:(6-20). Further preferably, the molar ratio of protamine to human serum albumin is 1:2.75-5.5. This technical solution can improve the stability and transfection efficiency of mRNA in vivo.

The present invention further provides a use of the mRNA-GalNAc targeting molecule as described above in preparation of an mRNA drug for specific drug delivery.

The present invention further provides a use of an mRNA-GalNAc targeting molecule as described above in targeted drug delivery in vivo. The 3′-end of the mRNA-GalNAc targeting molecule is linked to an N-acetylgalactosamine, and the mRNA-GalNAc targeting molecule specifically binds to sialoglycoprotein receptors on a surface of the liver cell through the N-acetylgalactosamine, which induces endocytosis and therefore allows the mRNA to enter into the cell for expression.

The mRNA-GalNAc targeting molecule of the present invention can be used in a drug delivery system. The 3′-end of the mRNA targeting molecule is linked to an N-acetylgalactosamine, and the mRNA-GalNAc targeting molecule specifically binds to sialoglycoprotein receptors on a surface of the liver cell through the N-acetylgalactosamine, which induces endocytosis and therefore allows the mRNA to enter into the cell for expression. In this way, the technical problem of targeting delivery of nucleic acid drugs during drug delivery is solved.

Compared with the prior art, the present invention has the following beneficial effects.

The technical solutions of the present invention achieve in vivo targeting delivery under the action of the positively charged proteins by directly linking the mRNA molecule expressing the target gene to the PolyA sequence coupled with GalNAc to synthesize the mRNA molecule with GalNAc at 3′-end, thereby realizing the purpose of targeted drug delivery in liver. The method of the present invention does not rely on traditional vectors such as liposomes and lipid nanoparticles, and is simpler and more reliable, solving the existing problem of coupling an mRNA to N-acetylgalactosamine and in vivo targeting delivery. Further, the efficiency and accuracy of linking can be greatly improved by the splint DNA. The positively charged protein molecule is used to neutralize the negative charge of the mRNA molecule itself to improve the stability of the mRNA-GalNAc in vivo and the efficiency of escape from endosome, thereby achieving improved tissue and organ targeting delivery of drugs in vivo.

### BRIEF DESCRIPTION OF THE DRAWINGS

FIG. 1 is a schematic flow chart showing the method for preparing an mRNA-GalNAc targeting molecule according to Example 1 of the present invention, wherein (a) is a structural schematic diagram showing the DNA fragment in the plasmid DNA and the mRNA molecule obtained by in vitro transcription; (b) is a schematic diagram showing the mRNA molecule and the PolyA modified with an N-acetylgalactosamine at 3′-end in a to-be-linked configuration; and (c) is a schematic diagram of the obtained mRNA-GalNAc targeting molecule.

FIG. 2 is a schematic flow chart showing the method for preparing an mRNA-GalNAc targeting molecule according to Example 2 of the present invention, wherein (a) is a structural schematic diagram showing the DNA fragment in the plasmid DNA and the mRNA molecule obtained by in vitro transcription; (b) is a schematic diagram showing the mRNA molecule linked to the PolyA modified with an N-acetylgalactosamine at 3′-end through the splint DNA; and (c) is a schematic diagram of the obtained mRNA-GalNAc targeting molecule.

FIG. 3 is a principle diagram showing preparation of the in vivo delivery system by interaction of the mRNA-GalNAc targeting molecule and the positively charged protein according to Example 2 of the present invention.

FIG. 4 is a principle schematic diagram showing the in vivo delivery of the mRNA-GalNAc targeting molecule according to Example 2 of the present invention, wherein by binding to ASGPR on a surface of liver cells and inducing endocytosis, the GalNAc-mRNA conjugate allows the mRNA to enter into the cells and express the target gene protein after escaping from endosome and ribosomal translation.

FIG. 5 is a diagram showing comparison results in term of delivering the target gene to a liver tissue in vivo by the GalNAc-mRNA delivery systems according to Example 3 of the present invention, wherein (a) is the activity comparison result of using the delivery systems with different ratios of protamine to human serum albumin for delivering luciferase (Luc) in vivo; (b) is the comparison result of the delivery systems with different ratios of protamine to human serum albumin in term of erythropoietin (Epo); and (c) is the comparison result of animals' hematocrit obtained after using the delivery systems with different ratios of protamine to human serum albumin for delivering erythropoietin (Epo).

FIG. 6 is a comparison schematic diagram showing expressions of green fluorescent protein (GFP) in liver cells obtained by the GalNAc-mRNA liver delivery systems with two different ratios of protamine to human serum albumin and a control delivery system without the positively charged proteins, according to Example 3 of the present invention.

FIG. 7 is a schematic diagram showing results of delivering luciferase (Luc) to the liver tissue in vivo by the GalNAc-mRNA liver delivery systems with different ratios of protamine to human serum albumin and the control delivery system, according to Example 4 of the present invention.

FIG. 8 shows effect of the Kozak sequence and the GalNAc modification on expression efficiency of the mRNA according to Example 5 of the present invention, wherein A shows the expression efficiency after the cells are transfected with the EPO mRNAs with and without the Kozak sequence; and B shows the expression efficiency after intramuscularly injecting Luciferase mRNAs with and without GalNAc modification into mice.

FIG. 9 is a concentration comparison of EPO proteins in blood obtained after intravenously injecting the EPO mRNAs containing different chemically modified bases into mice, according to Example 6 of the present invention.

### DETAILED DESCRIPTION OF THE INVENTION

The preferred Examples of the present invention will be described in further detail below.

### Example 1

Provided herein is an mRNA-GalNAc targeting molecule, which comprises an mRNA molecule that is linked to PolyA modified with an N-acetylgalactosamine at 3′-end, wherein a sequence of the mRNA molecule comprises a 5′ cap and a target gene sequence.

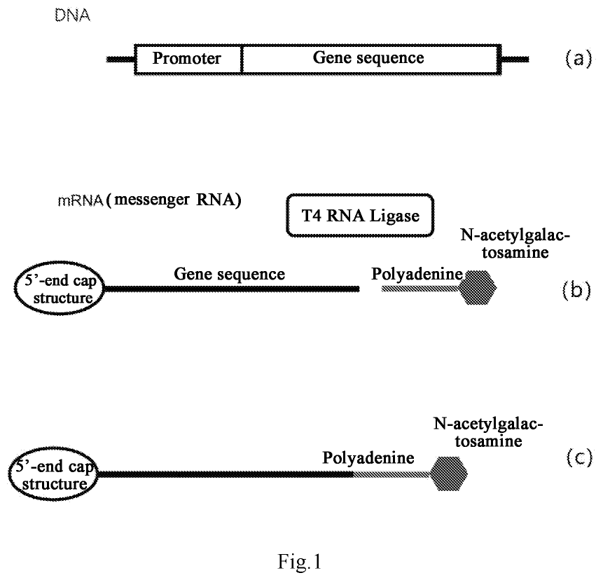

As shown in FIG. 1, the mRNA-GalNAc targeting molecule is prepared by the following steps.

Step S1, designing and synthesizing a plasmid vector having a promoter sequence and a target gene sequence;

Step S2, in vitro transcribing the plasmid vector of Step S1 as a template to obtain an mRNA molecule, wherein the sequence of the mRNA molecule comprises a 5′cap and a target gene sequence; and

Step S3, binding the mRNA molecule to the PolyA sequence modified with an N-acetylgalactosamine at 3′-end under the action of a ligase, to obtain the mRNA-GalNAc targeting molecule.

Further, the ligase is a DNA T4 ligase.

In this Example, a sequence of the promoter is as shown in SEQ ID No. 3.

Further, a sequence of the target gene is as shown in SEQ ID No. 4.

SEQ ID No. 3:
taatacgactcactatagg;
SEQ ID No. 4:
gtgagcaagggcgaggagaccacaatgggcgtaatcaagcccgacatgaagatcaagctgaagatg
gagggcaacgtgaatggccacgccttcgtgatcgagggcgagggcgagggcaagccctacgacggcacca
acaccatcaacctggaggtgaaggagggagcccccctgcccttctcctacgacattctgaccaccgcgttcagt
tacggcaacagggccttcaccaagtaccccgacgacatccccaactacttcaagcagtccttccccgagggct
actcttgggagcgcaccatgaccttcgaggacaagggcatcgtgaaggtgaagtccgacatctccatggagg
aggactccttcatctacgagatacacctcaagggcgagaacttcccccccaacggccccgtgatgcagaagg
agaccaccggctgggacgcctccaccgagaggatgtacgtgcgcgacggcgtgctgaagggcgacgtcaa
gatgaagctgctgctggagggcggcggccaccaccgcgttgacttcaagaccatctacagggccaagaagg
cggtgaagctgcccgactatcactttgtggaccaccgcatcgagatcctgaaccacgacaaggactacaacaa
ggtgaccgtttacgagatcgccgtggcccgcaactccaccgacggcatggacgagctgtacaagtaa.

The mRNA-GalNAc targeting molecule prepared in this Example can be used in preparation of an mRNA drug for specific drug delivery. The 3′-end of the mRNA-GalNAc targeting molecule is linked to an N-acetylgalactosamine, and the mRNA-GalNAc targeting molecule specifically binds to sialoglycoprotein receptors on a surface of the liver cell through the N-acetylgalactosamine, which induces endocytosis and therefore allows the mRNA to enter into the cell for expression.

### Example 2

Provided herein is an mRNA-GalNAc targeting molecule comprising an mRNA molecule, wherein a sequence of the mRNA molecule comprises a 5′ cap and a target gene sequence; the mRNA molecule is linked to PolyA modified with an N-acetylgalactosamine at 3′-end through a splint DNA; and the mRNA-GalNAc targeting molecule comprises a positively charged protein molecule.

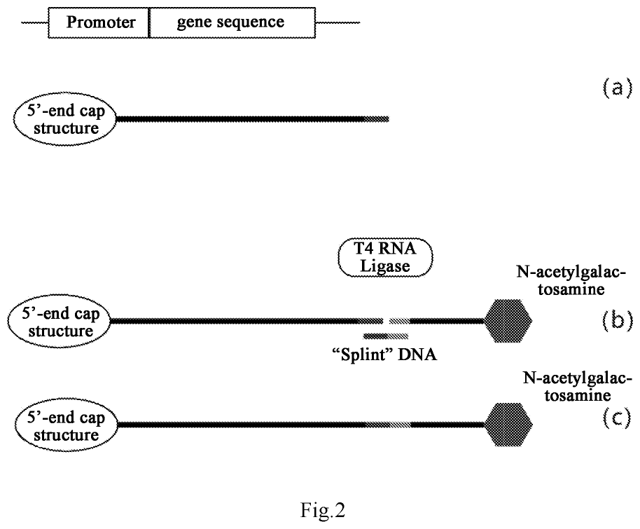

As shown in FIG. 2, the mRNA-GalNAc targeting molecule is prepared by the following steps.

Step S1, designing and synthesizing a plasmid vector having a promoter sequence, a target gene sequence and a sequence complementary to a first splint DNA sequence;

Step S2, in vitro transcribing the plasmid vector of Step S1 as a template to obtain an mRNA molecule, wherein a sequence of the mRNA molecule comprise a 5′cap, the target gene sequence, and an RNA sequence corresponding to the sequence complementary to the first splint DNA sequence, that are sequentially connected; and

Step S3, the PolyA fragment modified with an N-acetylgalactosamine at 3′-end has a 5′-end which is an RNA sequence that can be complementarily paired with a second splint DNA sequence, wherein the first splint DNA sequence and the second splint DNA sequence form the splint DNA; in annealing reaction, the mRNA molecule with the sequence complementary to the first splint DNA sequence and the PolyA molecule with the sequence complementarily paired with the second splint DNA sequence complementarily bind to the splint DNA, respectively; a 3′-end hydroxyl group of the mRNA molecule is linked to a 5′-end phosphate group of the PolyA modified with an N-acetylgalactosamine under the action of a T4 DNA ligase; and after treatment by a DNase, the mRNA targeting molecule modified with an N-acetylgalactosamine is obtained.

In this Example, a sequence of the first splint DNA is as shown in SEQ ID No. 1, and a sequence of the second splint DNA is as shown in SEQ ID No. 2.

A sequence of the promoter is as shown in SEQ ID No. 3.

A sequence of the target gene is as shown in SEQ ID No. 4.

Further, the ligase is a DNA T4 ligase.

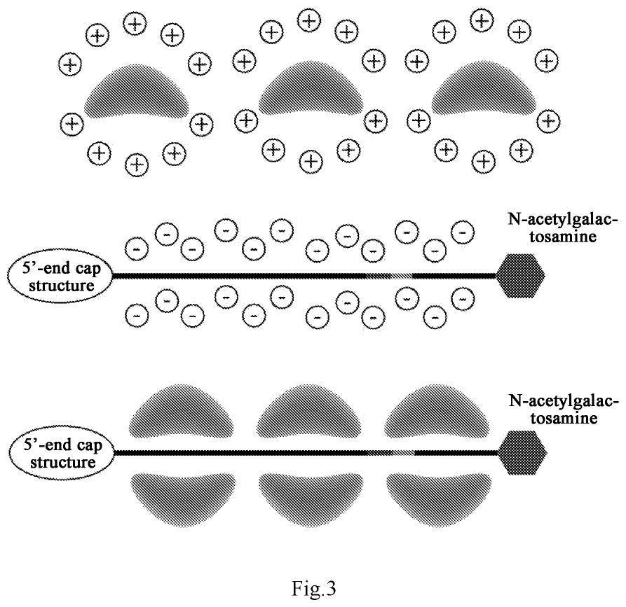

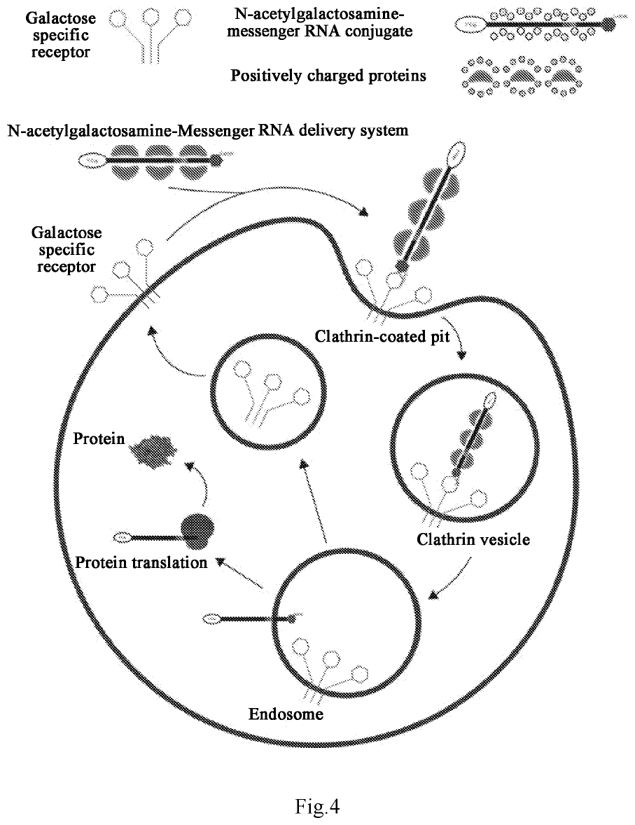

The mRNA-GalNAc targeting molecule obtained in this Example can be used in preparation of an mRNA drug for specific drug delivery. The 3′-end of the mRNA-GalNAc targeting molecule is linked to an N-acetylgalactosamine, and the mRNA-GalNAc targeting molecule specifically binds to sialoglycoprotein receptors on a surface of the liver cell through the N-acetylgalactosamine, which induces endocytosis and therefore allows the mRNA to enter into the cell for expression, as shown in FIGS. 3 and 4.

### Example 3

Provided herein is an in vivo delivery system, which comprises the mRNA-GalNAc targeting molecule of Example 2 and a positively charged protein molecule. After the mRNA-GalNAc targeting molecule was obtained, the in vivo delivery system was obtained by adjusting the charge property of the mRNA-GalNAc targeting molecule using adjuvants, protamine and/or human serum albumin (HSA). This step allowed the mRNA-GalNAc targeting molecule to contain a positively charged protein, which improved the stability and the transfection efficiency of the mRNA in vivo. Further, the positively charged protein was at least one of protamine and human serum albumin.

In this Example, by screening effective positively charged protein molecules and neutralizing the negative charge of the mRNA molecule itself, the stability of the mRNA-GalNAc in vivo was improved, the efficiency of escape from endosome was increased, thereby the tissue and organ targeting delivery in vivo was realized. The principle of action was as shown in FIG. 4.

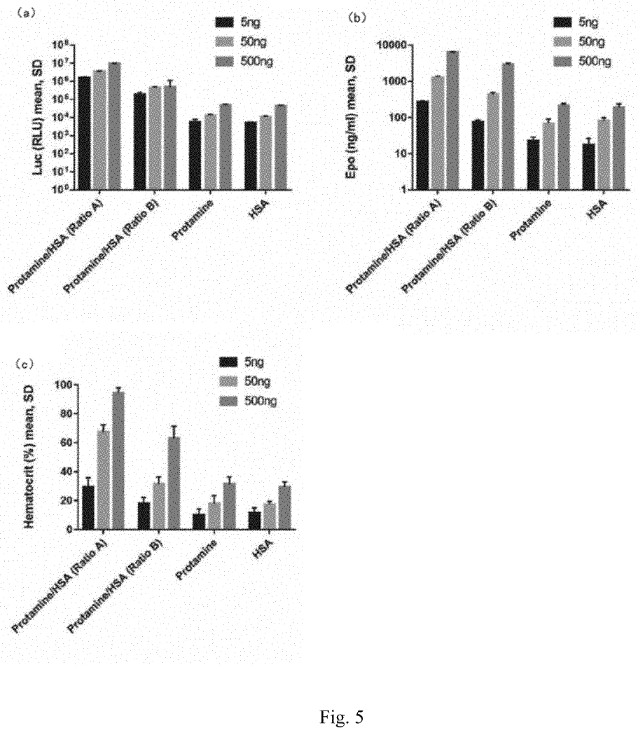

This Example also selected and used different ratios of protamine to human serum albumin, as well as protamine or human serum albumin alone for experiments. The molar Ratio A of protamine to HAS was 1:4, and the molar Ratio B of protamine to HAS was 1:12. The test results were as shown in FIGS. 5(a)-(c). It can be seen from comparison that, the delivery systems with the two mixed positively charged proteins of protamine and human serum albumin had better stability and transfection efficiency than the delivery systems with a single positively charged protein, and the delivery system with protamine and human serum albumin in Ratio A had the highest stability and transfection efficiency.

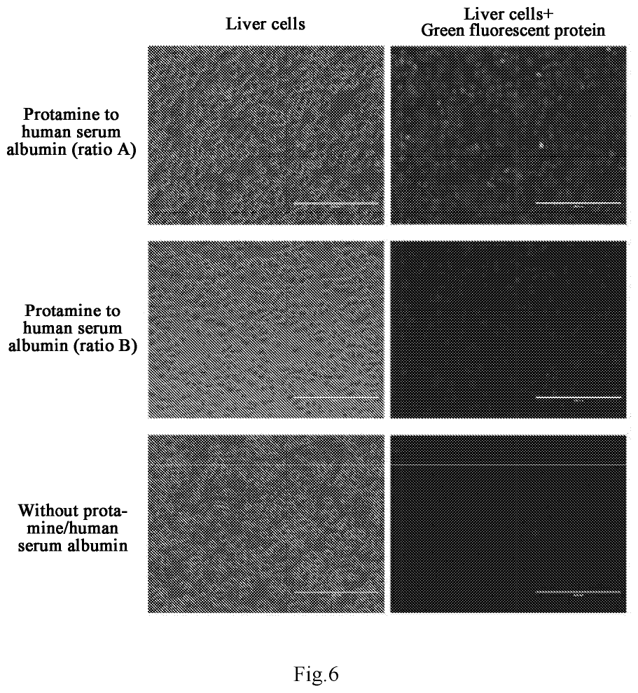

In addition, the delivery system with protamine and human serum albumin in Ratio A or Ratio B was compared experimentally to the delivery system without protamine and human serum albumin. As shown in FIG. 6, the delivery system with Ratio A or Ratio B was more efficient than the delivery system without protamine and human serum albumin in term of expression of green fluorescent protein (GFP) in liver cells.

### Example 4

Provided herein is an in vivo delivery system based on Example 3, which comprises an mRNA-GalNAc targeting molecule and a positively charged protein, wherein the target gene sequence in the mRNA-GalNAc targeting molecule is as shown in SEQ ID No. 5, which is different from Example 3.

SEQ ID No. 5:
atggaagatgccaaaaacattaagaagggcccagcgccattctacccactcgaagacgggaccgccg
gcgagcagctgcacaaagccatgaagcgctacgccctggtgcccggcaccatcgcctttaccgacgcacata
tcgaggtggacattacctacgccgagtacttcgagatgagcgttcggctggcagaagctatgaagcgctatgg
gctgaatacaaaccatcggatcgtggtgtgcagcgagaatagcttgcagttcttcatgcccgtgttgggtgccct
gttcatcggtgtggctgtggccccagctaacgacatctacaacgagcgcgagctgctgaacagcatgggcatc
agccagcccaccgtcgtattcgtgagcaagaaagggctgcaaaagatcctcaacgtgcaaaagaagctaccg
atcatacaaaagatcatcatcatggatagcaagaccgactaccagggcttccaaagcatgtacaccttcgtgact
tcccatttgccacccggcttcaacgagtacgacttcgtgcccgagagcttcgaccgggacaaaaccatcgccct
gatcatgaacagtagtggcagtaccggattgcccaagggcgtagccctaccgcaccgcaccgcttgtgtccga
ttcagtcatgcccgcgaccccatcttcggcaaccagatcatccccgacaccgctatcctcagcgtggtgccattt
caccacggcttcggcatgttcaccacgctgggctacttgatctgcggctttcgggtcgtgctcatgtaccgcttcg
aggaggagctattcttgcgcagcttgcaagactataagattcaatctgccctgctggtgcccacactatttagcttc
ttcgctaagagcactctcatcgacaagtacgacctaagcaacttgcacgagatcgccagcggcggggcgccg
ctcagcaaggaggtaggtgaggccgtggccaaacgcttccacctaccaggcatccgccagggctacggcct
gacagaaacaaccagcgccattctgatcacccccgaaggggacgacaagcctggcgcagtaggcaaggtg
gtgcccttcttcgaggctaaggtggtggacttggacaccggtaagacactgggtgtgaaccagcgcggcgag
ctgtgcgtccgtggccccatgatcatgagcggctacgttaacaaccccgaggctacaaacgctctcatcgacaa
ggacggctggctgcacagcggcgacatcgcctactgggacgaggacgagcacttcttcatcgtggaccggct
gaagagcctgatcaaatacaagggctaccaggtagccccagccgaactggagagcatcctgctgcaacaccc
caacatcttcgacgccggggtcgccggcctgcccgacgacgatgccggcgagctgcccgccgcagtcgtcg
tgctggaacacggtaaaaccatgaccgagaaggagatcgtggactatgtggccagccaggttacaaccgcca
agaagctgcgcggtggtgttgtgttcgtggacgaggtgcctaaaggactgaccggcaagttggacgcccgca
agatccgcgagattctcattaaggccaagaagggcggcaagatcgccgtgtaa.

After the mRNA-GalNAc targeting molecule was obtained, the in vivo delivery system was obtained by adjusting the charge property of the mRNA-GalNAc targeting molecule using adjuvants, protamine and HSA. The molar Ratio A of protamine to HAS was 1:4 and the molar Ratio B of protamine to HAS was 1:12.

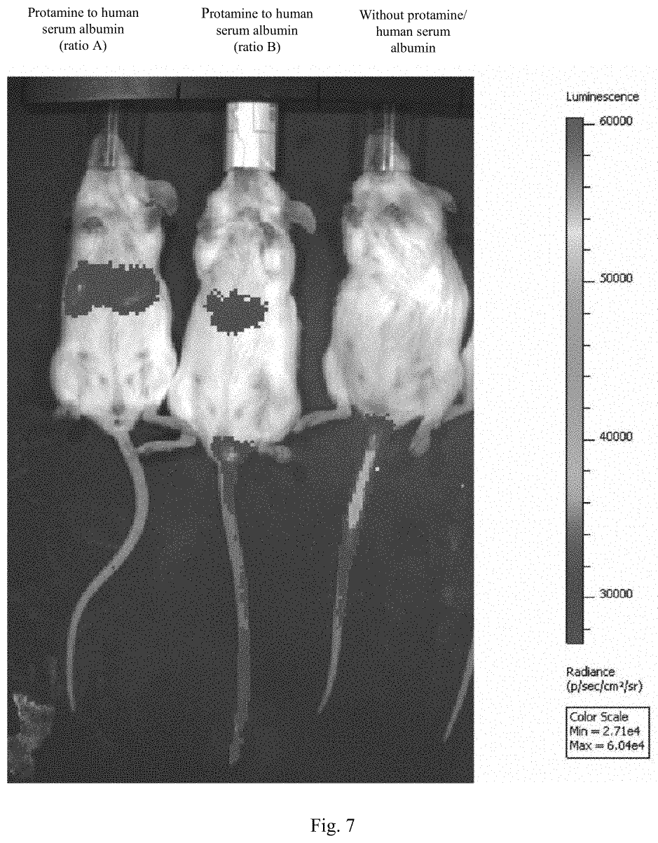

The delivery system with protamine and human serum albumin in Ratio A or Ratio B was compared experimentally to the delivery system without protamine and human serum albumin. As shown in FIG. 7, the delivery system with Ratio A or Ratio B was more efficient than the delivery system without protamine and human serum albumin in term of delivery of luciferase (Luc) to a liver tissue. The delivery system with protamine and human serum albumin in Ratio A had the best effect.

### Example 5

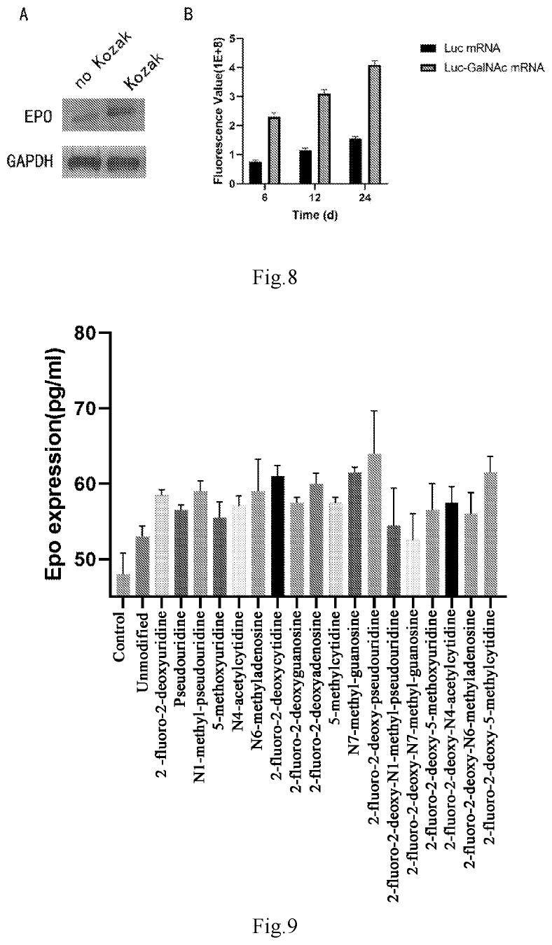

The EPO mRNAs with and without the Kozak sequence, as well as Luciferase mRNAs with and without GalNAc modification, were synthesized. 293T cells were cultured in vitro and transfected with the EPO mRNA, and the total protein in the 293T cells was extracted after 24 hours. The effect of the Kozak sequence on expression efficiency of the EPO mRNA was determined by Western blot assay. The Luciferase mRNA was injected into mice intramuscularly and after 24 hours, 200 ul of 15 mg/ml luciferin was injected intraperitoneally for three-dimensional color imaging. The effect of GalNAc modification on expression efficiency of the mRNA was compared based on luminescence intensities. The result was shown in FIG. 8.

### Example 6

The Epo mRNA-GalNAc molecules were synthesized. The unmodified versions were consisted of uridine, cytosine, adenosine, guanosine and chemically modified nucleosides. The modified versions were those in which the original nucleotide molecule was replaced with one of 2-fluoro-2-deoxyadenosine, 2-fluoro-2-deoxyuridine, 2-fluoro-2-deoxycytidine, 2-fluoro-2-deoxyguanosine, 2-fluoro-2-deoxy-5-methylcytidine, 2-fluoro-2-deoxy-pseudouridine, 2-fluoro-2-deoxy-N1-methyl-pseudouridine, 2-fluoro-2-deoxy-N7-methyl-guanosine, 2-fluoro-2-deoxy-5-methoxyuridine, 2-fluoro-2-deoxy-N4-acetylcytidine, 2-fluoro-2-deoxy-N6-methyladenosine, 5-methylcytidine, pseudouridine, N1-methyl-pseudouridine, N7-methyl-guanosine, 5-methoxyuridine, N4-acetylcytidine and N6-methyladenosine.

All of the mRNA molecules were formulated into a 250 ug/ml solution in physiological saline. Each 8-week-old Balb/c mouse was injected with 200 ul of the solution through the tail vein. The peripheral blood serum of the mouse was collected after 24 hours for Elisa experiment to determine expression level of the Epo protein.

The experimental protocol was as follows.

Reagents: Anti-EPO antibody (ab226956), and Goat Anti-Rabbit IgG H&L (HRP) (ab6721).

Consumable materials: Greiner 96-well microtiter plates, pipette tips 25 ml, pipette tips 1 ml, and pipette tips 300 ul.

Instruments and equipment: biotek Epch2 microplate reader, and Microplate 50TS Automatic Plate Washer.

### Experimental Procedures

Coating: The volume of the coating sample was 100 ul, and the mass of the coating standards was 2 ng/0.2 ng/0.02 ng/0.002 ng/0.0002 ng/0.00002 ng. A calculated amount of sample was taken and diluted in 100 ul of coating buffer, and then was added to the 96-well plate by multichannel pipettes. And then, the plate was covered with a microplate sealer and was allowed to stand at 4° C. overnight.

Blocking: The coated 96-well plate was tilted to remove the coating buffer. Then the coated 96-well plate was placed upside down on an absorbent paper and shook until there was no residue in the wells.

Plate washing: The washing buffer was prepared and diluted with deionized water by 50 folds, and added to the liquid inlet bottle of the plate washer. The program was set, in which the volume of the washing buffer in each well was set to 300 μl and the washing was repeated four times.

Blocking: The washed plate was placed upside down and shook to remove the solution inside until dryness. Then the plate was added with the blocking buffer in a volume of 250 ul per well and covered with a microplate sealer, and allowed to stand at room temperature for 2 hours.

Plate washing: The blocked microtiter plate was washed according to step 3.

Incubation of Primary antibody: The primary antibody was diluted with a dilution buffer and added to the washed 96-well plate in a volume of 100 ul per well. The plate was covered with a microplate sealer and incubated at room temperature for 1.5 hours.

Plate washing: The plate was washed according to step 3, and the washing was repeated 6 times.

Addition of secondary antibody: HRP-labeled goat anti-rabbit IgG was diluted with the dilution buffer at dilution factor of 10000 and the diluted antibody was added to the microtiter plate in a volume of 100 ul per well. The plate was covered with a microplate sealer and incubated for 1 hour at room temperature in the dark.

Plate washing: The plate was washed according to step 8. In this step, the plate must be washed thoroughly and placed upside down to remove the solution until dryness.

Color development: 100 ul of TMB buffer was added. The color development was performed in the dark for 20-30 minutes. At this time, the positive sample exhibited blue color.

Termination: 100 ul of a stop buffer was added. The reading was performed on the microtiter plate within 10 minutes, and the absorption wavelength was set to 450 nm.

The experimental results were as shown in FIG. 9. The chemical modification of bases can significantly increase the expression level of mRNA-GalNAc molecules.

The above contents are further detailed descriptions of the present invention in conjunction with specific preferred embodiments, and cannot be considered that specific embodiments of the present invention are limited thereto. For those of ordinary skill in the technical field to which the present invention belongs, several simple deductions or replacements can be made without departing from the concept of the present invention, all of which should be regarded as belonging to the protection scope of the present invention.

1-16. (canceled)

17. An mRNA-N-acetylgalactosamine (GalNAc) targeting molecule comprising an mRNA molecule and a GalNAc, wherein the mRNA molecule comprises a target gene sequence, and wherein the GalNAc is directly conjugated to a polyA sequence of the mRNA molecule.

18. The mRNA-GalNAc targeting molecule of claim 17, wherein the mRNA-GalNAc targeting molecule comprises a 5′ cap.

19. The mRNA-GalNAc targeting molecule of claim 18, wherein the 5′ cap comprises one or more of Cap0, Cap1, Cap2, ARCA, inosine, N1-methyl-guanosine, 2′fluoro-guanosine, 7-deaza-guanosine, 8-oxo-guanosine, 2-amino-guanosine, LNA-guanosine, 2-azido-guanosine, 7-methyl-guanosine-5′-triphosphate-5′-adenosine, guanosine-5′-triphosphate-5′-adenosine, 7-methyl-guanosine-5′-triphosphate-5′-guanosine, guanosine-5′-triphosphate-5′-guanosine, and 7-methyl-guanosine-5′-triphosphate-5′-2-methoxyadenine-guanosine.

20. The mRNA-GalNAc targeting molecule of claim 17, wherein the mRNA molecule comprises one or more of a chemically modified nucleoside, wherein the chemically modified nucleoside comprises 2-fluoro-2-deoxyadenosine, 2 -fluoro-2-deoxyuridine, 2-fluoro-2-deoxycytidine, 2-fluoro-2-deoxyguanosine, 2-fluoro-2-deoxy-5-methylcytidine, 2-fluoro-2-deoxy-pseudouridine, 2-fluoro-2-deoxy-N1-methyl-pseudouridine, 2-fluoro-2-deoxy-N7-methyl-guanosine, 2-fluoro-2-deoxy-5-methoxyuridine, 2-fluoro-2-deoxy-N4-acetylcytidine, 2-fluoro-2-deoxy-N6-methyladenosine, 5-methylcytidine, pseudouridine, N1-methyl-pseudouridine, N7-methyl-guanosine, 5-methoxyuridine, N4-acetylcytidine or N6-methyl adenosine.

21. The mRNA-GalNAc targeting molecule of claim 17, wherein the mRNA molecule comprises a 5′ UTR and/or a 3′ UTR.

22. The mRNA-GalNAc targeting molecule of claim 21, wherein the 5′ UTR comprises a Kozak sequence.

23. An in vivo delivery system comprising the mRNA-GalNAc targeting molecule of claim 17 and a positively charged protein molecule.

24. The in vivo delivery system of claim 23, wherein the positively charged protein molecule comprises protamine and/or human serum albumin.

25. The in vivo delivery system of claim 24, wherein the positively charged protein molecule comprises protamine and human serum albumin, and wherein a molar ratio of the protamine to the human serum albumin is 1: (2.75-5.5) or 1: (6-20).

26. A pharmaceutical composition comprising the mRNA-GalNAc targeting molecule of claim 17, or the in vivo delivery system of any one of claim 23, and a pharmaceutically acceptable excipient.

27. A method of preparing an mRNA-N-acetylgalactosamine (GalNAc) targeting molecule comprising an mRNA molecule and a GalNAc, wherein the method comprises:
(a). in vitro transcribing from a plasmid vector the mRNA molecule, wherein the plasmid vector comprises a promoter sequence and a target gene sequence, and wherein the mRNA is transcribed from the target gene sequence; and
(b). conjugating the mRNA molecule and a polyA sequence modified with a GalNAc at 3′-end with a ligase,
thereby obtaining the mRNA-GalNAc targeting molecule.

28. The method of claim 27, wherein the plasmid vector comprises a 5′ UTR and/or a 3′ UTR, and optionally wherein the 5′ UTR comprises a Kozak sequence.

29. The method of claim 27, wherein the promoter is T3, T7, or SP6, and optionally wherein the promoter comprises a sequence set forth in SEQ ID NO: 3.

30. The method of claim 27, wherein (a) further comprises processing the mRNA molecule with 5′ capping, and optionally wherein the 5′ capping comprises adding one or more of Cap0, Cap1, Cap2, ARCA, inosine, N1-methyl-guanosine, 2′fluoro-guanosine, 7-deaza-guanosine, 8-oxo-guanosine, 2-amino-guanosine, LNA-guanosine, 2-azido-guanosine, 7-methyl-guanosine-5′-triphosphate-5′-adenosine, guanosine-5′-triphosphate-5′-adenosine, 7-methyl-guanosine-5′-triphosphate-5′-guanosine, guanosine-5′-triphosphate-5′-guanosine, and 7-methyl-guanosine-5′-triphosphate-5′-2-methoxyadenine-guanosine.

31. The method of claim 27, wherein (a) further comprising modifying the mRNA molecule with one or more of a chemically modified nucleoside, wherein the chemically modified nucleoside comprises 2-fluoro-2-deoxyadenosine, 2-fluoro-2-deoxyuridine, 2-fluoro-2-deoxycytidine, 2-fluoro-2-deoxyguanosine, 2-fluoro-2-deoxy-5-methylcytidine, 2-fluoro-2-deoxy-pseudouridine, 2-fluoro-2-deoxy-N1-methyl-pseudouridine, 2-fluoro-2-deoxy-N7-methyl-guanosine, 2-fluoro-2-deoxy-5-methoxyuridine, 2-fluoro-2-deoxy-N4-acetylcytidine, 2-fluoro-2-deoxy-N6-methyladenosine, 5-methylcytidine, pseudouridine, N1-methyl-pseudouridine, N7-methyl-guanosine, 5-methoxyuridine, N4-acetylcytidine or N6-methyl adenosine.

32. The method of claim 27, wherein the ligase is a T4 ligase.

33. The method of claim 27, wherein in (b) a 3′-end hydroxyl group of the mRNA molecule is linked to a 5′-end phosphate group of the polyA sequence.

34. A method of preparing an mRNA-N-acetylgalactosamine (GalNAc) targeting molecule comprising an mRNA molecule and a GalNAc, wherein the method comprises:
(a). in vitro transcribing from a plasmid vector the mRNA molecule, wherein the plasmid vector comprises a promoter sequence, a target gene sequence, and a sequence that is complementary to a first splint DNA sequence, and wherein the mRNA is transcribed from the target gene sequence;
(b). providing a moiety, wherein the moiety comprises, from its 5′end to 3′ end, an RNA sequence complementary to a second splint DNA sequence, a polyA sequence, and the GalNAc; and
(c). conjugating the mRNA molecule and the moiety with a ligase and a splint DNA, and the splint DNA comprises the first splint DNA sequence and the second splint DNA sequence,
thereby obtaining the mRNA-GalNAc targeting molecule.

35. The method of claim 34, wherein the first splint DNA sequence comprises a sequence set forth in SEQ ID NO: 1.

36. The method of claim 34, wherein the second splint DNA sequence comprises a sequence set forth in SEQ ID NO: 2.

37. The method of claim 34, wherein the plasmid vector comprises a 5′ UTR and/or a 3′ UTR, and optionally wherein the 5′ UTR comprises a Kozak sequence.

38. The method of claim 34, wherein the promoter is T3, T7, or SP6, and optionally wherein the promoter comprises a sequence set forth in SEQ ID NO: 3.

39. The method of claim 34, wherein (a) further comprises processing the mRNA molecule with 5′ capping, and optionally wherein the 5′ capping comprises adding one or more of Cap0, Cap1, Cap2, ARCA, inosine, N1-methyl-guanosine, 2′fluoro-guanosine, 7-deaza-guanosine, 8-oxo-guanosine, 2-amino-guanosine, LNA-guanosine, 2-azido-guanosine, 7-methyl-guanosine-5′-triphosphate-5′-adenosine, guanosine-5′-triphosphate-5′-adenosine, 7-methyl-guanosine-5′-triphosphate-5′-guanosine, guanosine-5′-triphosphate-5′-guanosine, and 7-methyl-guanosine-5′-triphosphate-5′-2-methoxyadenine-guanosine.

40. The method of claim 34, wherein (a) further comprising modifying the mRNA molecule with one or more of 2-fluoro-2-deoxyadenosine, 2-fluoro-2-deoxyuridine, 2-fluoro-2-deoxycytidine, 2-fluoro-2-deoxyguanosine, 2-fluoro-2-deoxy-5-methylcytidine, 2-fluoro-2-deoxy-pseudouridine, 2-fluoro-2-deoxy-N1-methyl-pseudouridine, 2-fluoro-2-deoxy-N7-methyl-guanosine, 2-fluoro-2-deoxy-5-methoxyuridine, 2-fluoro-2-deoxy-N4-acetylcytidine, 2-fluoro-2-deoxy-N6-methyladenosine, 5-methylcytidine, pseudouridine, N1-methyl-pseudouridine, N7-methyl-guanosine, 5-methoxyuridine, N4-acetylcytidine and N6-methyladenosine.

41. The method of claim 34, wherein the ligase is a T4 ligase.

42. The method of claim 34, wherein (c) is carried out in an annealing reaction.

43. The method of claim 34, wherein in (c) a 3′-end hydroxyl group of the mRNA molecule is linked to a 5′-end phosphate group of the polyA sequence.

44. A method of expressing a target gene sequence in a subject, wherein the method comprises delivering the mRNA-GalNAc targeting molecule of any one of claims 1 to 22, or the in vivo delivery system of any one of claims 23 to 25, or the pharmaceutical composition of claim 26.

45. The method of claim 44, wherein the delivering is carried out intravenously or intramuscularly.
